(19) United States
(12) Patent Application Publication
(10) Pub. No.: US 2020/0000729 A1
(43) Pub. Date: Jan. 2, 2020

(54) ERYTHRITOL GRANULES AND METHOD FOR PRODUCING SAME, METHOD FOR PRODUCING TABLETS USING SAME, AND TABLETS

(71) Applicant: B FOOD SCIENCE CO., LTD., Chita-shi, Aichi (JP)

(72) Inventors: Yuki KIMURA, Chita-shi, Aichi (JP); Takumi TOCHIO, Chita-shi, Aichi (JP); Kanako MINODA, Chita-shi, Aichi (JP); Hiromitsu YAMAMOTO, Nagoya-shi, Aichi (JP)

(21) Appl. No.: 16/489,027

(22) PCT Filed: Feb. 28, 2018
(86) PCT No.: PCT/JP2018/007497

§ 371 Date: Aug. 27, 2019

(30) Foreign Application Priority Data
Feb. 28, 2017 (JP) 2017-037138

(51) Int. Cl. A61K 9/20; A61K 9/16

(52) U.S. Cl. CPC A61K 9/2018; A61K 9/2054; A61K 9/2077; A61K 9/1652; A61K 9/1694; A61K 9/2013

(57) ABSTRACT

[Problem]

To provide: erythritol granules which have sufficient bindability while retaining the characteristic properties of erythritol; a method for producing the erythritol granules; a method for producing tablets using the erythritol granules; and tablets.

[Solution]

Erythritol granules each containing hydroxypropyl cellulose or hydroxypropyl methylcellulose, said erythritol granules having such a property that, when a sample prepared by adding 1.6 mg of magnesium stearate to 160 mg of the erythritol granules is filled in a mortar having a diameter of 8 mm and then the sample is compressed at a compression rate of 10 mm/min under a pressure of 0 to 100 MPa, the average yield pressure at 30 to 100 MPa is less than 2941 MPa. According to the present invention, it becomes possible to produce erythritol granules which have sufficient bindability while retaining the characteristic properties of erythritol.

### TECHNICAL FIELD

The present invention relates to granules (erythritol granules) containing erythritol as a main ingredient, and more particularly to erythritol granules preferable for producing tablets by a dry direct tableting method and a method for producing the granules, a method for producing tablets using the granules, and tablets.

### BACKGROUND ART

Erythritol is a sugar alcohol having light taste without continuity of sweet taste and having favorable sweetness like sugar. Moreover, erythritol has useful properties of being calorie-free and non-cariogenic, having relatively small laxative action, exerting no influence on the blood sugar level, having a flavoring effect of suppressing unfavorable taste such as bitterness and grassy smell, etc., and therefore, it has been expected to be utilized as an excipient in the production of tablets of medicines, supplements and the like.

On the other hand, methods for producing tablets (tableting methods) include a “dry direct tableting method (direct tableting method)” in which an ingredient such as a medicinal ingredient and an additive such as an excipient are mixed and the resulting mixture is tableted as it is without adding water and a “wet granulation tableting method” in which a mixture of a medicine and an additive is granulated using a binder solution or an appropriate solvent such as water and the resulting granules are dried and then tableted. Of these, the former has advantages that it can be applied even when the medicinal ingredient or the like is not resistant to water, the process control is easy owing to simple steps, and the product manufacturing cost can be also reduced, and hence, cases adopting this method are increasing in recent years.

In this regard, however, the above-described erythritol has strong crystallizability and low hygroscopicity, so that it has a problem that the binding property required when tableting is carried out by the direct tableting method is low. Then, granules for tableting which contain erythritol as a main ingredient and have binding property have been studied and developed, and for example, in Patent Literature 1, a method for producing a granulate, including granulating a composition containing erythritol and a saccharified reduced starch by kneading and extrusion molding is disclosed, in Patent Literature 2, a method for producing a granulate, including placing erythritol powder in a fluidized bed granulation coating apparatus and spraying an erythritol solution is disclosed, and in Patent Literature 3, a method for producing erythritol spherical granules for direct tableting, including granulating erythritol ultrafine powder having an average granule diameter of 0.4 μm to 23 μm is disclosed.

### CITATION LIST

### Patent Literature

Patent Literature 1: Japanese Patent No. 2852498

Patent Literature 2: Japanese Patent No. 3491887

Patent Literature 3: Japanese Patent No. 6061768

### SUMMARY OF INVENTION

### Technical Problem

However, the method described in Patent Literature 1 uses reduced starch syrup, reduced maltose starch syrup or the like, and therefore, granules for tableting which utilize characteristics of erythritol, such as calorie-free property and property of no influence on the blood sugar level, cannot be produced. The method described in Patent Literature 2 needs a step of sifting a granulate with a sieve to selectively recover granules having granule sizes in a prescribed range ([Claim 1], paragraphs [0023] and [0039]), and the number of steps increases or the product yield decreases, so that there is concern about an increase in the manufacturing cost. The method described in Patent Literature 3 is a method for producing granules by the use of only erythritol without using a binder or the like, and according to the knowledge of the present inventors, it is thought that granules for tableting which have sufficient binding property cannot be produced. Accordingly, development of erythritol granules for tableting which have sufficient binding property while retaining characteristics of erythritol and a method for producing the erythritol granules for tableting more simply and efficiently has been desired.

The present invention has been made in order to solve such problems, and it is an object of the present invention to provide erythritol granules having sufficient binding property while retaining characteristics of erythritol and employable for producing tablets by a direct tableting method and a method for producing the granules, a method for producing tablets using the granules, and tablets.

### Solution to Problem

The present inventors have earnestly studied, and as a result, they have found that erythritol has good compatibility with hydroxypropyl cellulose (HPC) or hydroxypropyl methylcellulose (HPMC), and by carrying out granulation using these, granules having properties preferable for producing tablets by a direct tableting method and retaining characteristics of erythritol can be simply and efficiently produced. Then, the present inventors have completed the respective inventions described below based on this knowledge.

(1) Erythritol granules according to a first embodiment of the present invention are erythritol granules comprising HPC or HPMC, the granules having a property that when a sample obtained by adding 1.6 mg of magnesium stearate to 160 mg of the erythritol granules is filled in a mortar having a diameter of 8 mm and compressed at a compression rate of 10 mm/min and a pressure of 0 to 100 MPa, an average yield pressure in the range of 30 to 100 MPa is less than 2941 MPa.

(2) Erythritol granules according to a second embodiment of the present invention comprise more than 1.48% by mass and less than 15.25% by mass of HPC or more than 1.48% by mass and less than 10.71% by mass of HPMC.

(3) The erythritol granules according to the present invention can be preferably used for producing tablets by a dry direct tableting method (direct tableting method).

(4) The erythritol granules according to the present invention preferably have a property that when tablets each having a diameter of 8 mm and weighing 200 mg per tablet are formed by adding 1 part by weight of magnesium stearate to 100 parts by weight of the erythritol granules and then tableting the mixture by a direct tableting method at a tableting pressure of 5.0 to 6.0 kN, a hardness of each of the tablets is not less than 3.5 kgf.

(5) A method for producing erythritol granules according to the present invention comprises a granulation step of spraying a spray liquid comprising HPC and/or HPMC to a erythritol powder while fluidizing or stirring the erythritol powder, and then carrying out drying.

(6) In the method for producing erythritol granules according to the present invention, the granulation step is preferably carried out by a fluidized bed granulation method.

(7) In the method for producing erythritol granules according to the present invention, the spray liquid preferably contains HPC in a concentration of more than 2.5% by mass and less than 30% by mass, or preferably contains HPMC in a concentration of more than 2.5% by mass and less than 20% by mass.

(8) In the method for producing erythritol granules according to the present invention, the spray liquid preferably further comprises erythritol.

(9) In the method for producing erythritol granules according to the present invention, it is preferable that in the spray liquid, the erythritol be contained in a concentration of less than 35% by mass and a weight ratio between the erythritol and HPC be either the following (a) or (b), or a weight ratio between the erythritol and HPMC be either the following (c) or (d): (a) more than 2.5 parts by weight and less than 30 parts by weight of HPC based on 33 parts by weight of the erythritol, (b) more than 3.3 parts by weight and less than 35 parts by weight of the erythritol based on 5 parts by weight of HPC, (c) more than 2.5 parts by weight and less than 20 parts by weight of HPMC based on 33 parts by weight of the erythritol, (d) more than 0 part by weight and less than 35 parts by weight of the erythritol based on 5 parts by weight of HPMC.

(10) A method for producing tablets according to the present invention comprises a tableting step of tableting a mixture of the erythritol granules according to the present invention and a medicinal ingredient or a food ingredient by a direct tableting method.

(11) A tablet according to the present invention comprises the erythritol granules according to the present invention and a medicinal ingredient or a food ingredient.

### Advantageous Effects of Invention

The erythritol granules according to the present invention have sufficient binding property while retaining characteristics of erythritol. According to the present invention, the erythritol granules can be simply and efficiently produced. By using the erythritol granules, tablets utilizing characteristics of erythritol can be simply produced by a direct tableting method.

### BRIEF DESCRIPTION OF DRAWINGS

FIG. 1 is a group of photographs each showing adhesion of a spray liquid to a opening of a nozzle of a granulation apparatus.

FIG. 2 is a group of electron microscopic observation images of samples in Example 3. It shows the observation images of ungranulated powdery erythritol (ungranulated ERT), erythritol granules containing no binder (No. 6), and erythritol granules containing hydroxypropyl methylcellulose (HPMC) (No. 2 and No. 8).

FIG. 3 is a group of views showing Heckel plots measured in Example 4, wherein the upper view shows Heckel plots of ungranulated ERT and erythritol granules (No. 2) obtained by carrying out granulation while spraying a spray liquid containing HPMC, and the lower view shows Heckel plots of ungranulated ERT and erythritol granules (No. 8) obtained by carrying out granulation while spraying a spray liquid containing HPMC and erythritol.

### DESCRIPTION OF EMBODIMENTS

The erythritol granules according to the present invention and the method for producing the granules, the method for producing tablets using the granules, and tablets are described in detail hereinafter.

In the present invention, a “tablet” refers to a molded product obtained by compression molding powder into a small-sized fixed shape. That is to say, in the tablets according to the present invention, not only medicines and quasi-drugs but also food and drink, such as health food (supplement, etc.) and confectionary (tablet confectionary), are included.

In the present invention, “erythritol granules” refers to granules containing erythritol as a main ingredient or an aggregate thereof, and they may be granules composed only of erythritol, or may be granules further containing ingredients other than erythritol. The granule diameters of the granules only need to be larger than the granule diameters of erythritol powder, and from the viewpoint of being used for producing tablets by a direct tableting method, the median diameter (d50) is preferably not less than 50 μm and less than 250 μm.

The present invention provides erythritol granules having properties preferable for producing tablets by a direct tableting method and retaining characteristics of erythritol. As shown in Example 3 described later, the erythritol granules according to the present invention each have a structure having many pores (porous structure), and therefore, they have tabletability, such as high formability and binding property in the tablet production and high hardness of tablets produced.

Here, as an indication to indicate tabletability of granules, an average yield pressure is known. As shown in Example 4 described later, the average yield pressure can be determined by making a plot (Heckel plot) of a relationship between a compression pressure (P) value given when the granules are filled in a mortar of a universal testing machine and compressed and a natural logarithm (In(1/ε)) value of a reciprocal number of a void ratio of a granule layer at the compression pressure. The average yield pressure is defined as a reciprocal number of a slope of a linear portion of the Heckel plot, and as the average yield pressure related to a stage in which the granule layer displays plastic deformation is lowered, plastic deformation easily occurs, that is, it indicates that the tabletability is high. When the erythritol granules of the present invention having a porous structure and having tabletability is expressed in terms of an average yield pressure, the following values can be given as examples: when a sample obtained by adding 1.6 mg of magnesium stearate (lubricant) to 160 mg of the erythritol is filled in a mortar having a diameter of 8 mm and compressed at a compression rate of 10 mm/min and a pressure of 0 to 100 MPa, the average yield pressure in the range of 30 to 100 MPa is around 1400 MPa, or not less than 1200 MPa and not more than 1600 MPa, not less than 1000 MPa and not more than 1800 MPa, not less than 800 MPa and not more than 2000 MPa, not less than 600 MPa and not more than 2200 MPa, not less than 400 MPa and not more than 2400 MPa, not less than 200 MPa and not more than 2600 MPa, not more than 2800 MPa, or not more than 2941 MPa.

That is to say, the erythritol granules according to a first embodiment of the present invention are erythritol granules comprising HPC or HPMC, the granules having a property that when a sample obtained by adding 1.6 mg of magnesium stearate to 160 mg of the erythritol granules is filled in a mortar having a diameter of 8 mm and compressed at a compression rate of 10 mm/min and a pressure of 0 to 100 MPa, the average yield pressure in the range of 30 to 100 MPa is less than 2941 MPa.

When the erythritol granules of the present invention having a porous structure and having tabletability are expressed in terms of a content of a binder therein, the content of HPC can be said to be more than 1.48% by mass and less than 5.25% by mass, and the content of HPMC can be said to be more than 1.48% by mass and less than 10.71% by mass. That is to say, the erythritol granules according to a second embodiment of the present invention are erythritol granules comprising HPC or HPMC, the granules comprising more than 1.48% by mass and less than 15.25% by mass of HPC, or more than 1.48% by mass and less than 10.71% by mass of HPMC.

A hardness of tablets produced by a direct tableting method using only erythritol granules without mixing a medicinal ingredient, a food ingredient or the like or using the erythritol granules mixed with only a lubricant also becomes an indication to indicate tabletability of the erythritol granules. When the erythritol granules of the present invention having a porous structure and having tabletability are expressed in terms of a hardness of tablets, the following values can be given as examples: when tablets each having a diameter of 8 mm and weighing 200 mg per tablet are formed by adding 1 part by weight of magnesium stearate (lubricant) to 100 parts by weight of the erythritol granules and then tableting the mixture by a direct tableting method at a tableting pressure of 5.0 to 6.0 kN, the hardness of each of the tablets is not less than 3.5 kgf.

The erythritol granules may contain other binders, sugar alcohols, and food additives or pharmaceutical additives, such as perfumes, colorants and preservatives as long as the features of the present invention are not impaired. Here, examples of other binders include methylcellulose, hydroxyethyl methylcellulose, pullulan, sodium alginate, agar, gelatin, carboxylmethylcellulose sodium, polyvinylpyrrolidone and polyvinyl alcohol.

Erythritol is a sugar alcohol having a chemical name of 1,2,3,4-butaneterol, and is also called erythritol. As powdery erythritol, commercially available one may be used, or one produced in accordance with a method publicly known to a person skilled in the art may be used. The publicly known production method is, for example, a method in which erythritol-producing microorganisms are cultured using glucose or the like as a carbon source to produce erythritol and the erythritol is refined. Here, examples of the erythritol-producing microorganisms include microorganisms belonging to genus Trigonopsis or genus Candida (Japanese Patent Publication No. 47-41549), microorganisms belonging to genus Torulopsis, genus Hansenula, genus Pichia or genus Debaryomyces (Japanese Patent Publication No. 51-21072), microorganisms belonging to genus Moniliella (Japanese Patent Laid-Open No. 60-110295, Japanese Patent Laid-Open No. 10-215887), microorganisms belonging to genus Aureobasidium (Japanese Patent Publication No. 63-9831), and microorganisms belonging to genus Yarrowia (Japanese Patent Laid-Open No. 10-215887). The culture can be carried out under common conditions suitable for each microorganism. For the refining of erythritol, steps of cell separation, fractionation of erythritol by chromatography, desalination, decolorization, crystallization, crystal decomposition and drying can be carried out in accordance with conventional methods.

Hydroxypropyl cellulose (HPC) is a cellulose derivative in which a hydroxypropoxyl group (—OCH₂CHOHCH₃) is introduced into a skeleton of cellulose. In the present invention, commercially available HPC can be used, and its viscosity, molecular weight, granule diameter, degree of molar substitution, content of a hydroxypropoxyl group, etc. can be appropriately determined according to the desired properties of the erythritol granules, the granulation method, etc. In Examples 1 to 5 described later, HPC (HPC SSL SFP, Nippon Soda Co., Ltd.) having a viscosity of 2 to 2.9 millipascals second (mPa·s) (20° C./2% aqueous solution), a molecular weight of about 40000 and a median diameter of 20 μm is used.

Hydroxypropyl methylcellulose (HPMC) is a cellulose derivative in which a methoxyl group (—OCH₃) and a hydroxypropoxyl group (—OCH₂CHOHCH₃) are introduced into a skeleton of cellulose. In the present invention, commercially available HPMC can be used, and its viscosity, molecular weight, granule diameter, degrees of substitution of methoxyl group and hydroxypropoxyl group, contents thereof, etc. can be appropriately determined according to the desired properties of the erythritol granules, the granulation method, etc. In Examples 1 to 5 described later, HPMC (TC-5, Shin-Etsu Chemical Co., Ltd.) having a viscosity of 3 to 15 mPa·s (20° C./2% aqueous solution), a methoxyl group content of 28.0 to 30.0% by mass (per dry weight) and a hydroxypropoxyl group content of 7.0 to 12.0% by mass (per dry weight) is used.

The erythritol granules can be produced by, for example, a granulation step of spraying a spray liquid comprising HPC and/or HPMC methylcellulose to erythritol powder while fluidizing or stirring the erythritol powder, and then carrying out drying. That is to say, the present invention also provides a method for producing erythritol granules, comprising the above step.

The granulation step can be carried out not only by a fluidized bed granulation method as shown in the test method (2) of the working examples described later but also by a stirring granulation method, a spray drying method or the like. Here, the fluidized bed granulation method is a method of wet granulation and is a method in which a hot air is fed from the lower part of a granulation room and blows up a raw material powder into the air to thereby form a layer wherein the granules are fluidized, and then a liquid (spray liquid) is sprayed to grow the raw material powder into a granulate (granules) through aggregation or coating. Granulation by the fluidized bed granulation method can be carried out using a commercially available granulation apparatus.

That is to say, when the granulation step is carried out by the fluidized bed granulation method, erythritol granules can be produced by spraying a spray liquid containing HPC and/or HPMC to a erythritol powder while stirring the erythritol powder with a hot air, and then carrying out drying with the hot air.

Examples of solvents for HPC and/or HPMC in the spray liquid include water, alcohols such as ethanol, and mixtures of them. To the spray liquid, other binders, sugar alcohols, and food additives or pharmaceutical additives, such as perfumes, colorants and preservatives, may be added as long as the features of the present invention are not impaired. Examples of other binders include methylcellulose, hydroxyethyl methylcellulose, pullulan, sodium alginate, agar, gelatin, carboxylmethylcellulose sodium, polyvinylpyrrolidone and polyvinyl alcohol.

The concentration of HPC in the spray liquid can be, for example, 2.5 to 30% by mass, 2.6 to 29% by mass, 2.7 to 28% by mass, 2.8 to 27% by mass, 2.9 to 26% by mass, 3.0 to 25% by mass, 3.1 to 24% by mass, 3.2 to 23% by mass, 3.3 to 22% by mass, 3.4 to 21% by mass, 3.5 to 21% by mass, or the like. The concentration of HPMC in the spray liquid can be, for example, 2.5 to 20% by mass, 2.6 to 19% by mass, 2.7 to 18% by mass, 2.8 to 17% by mass, 2.9 to 16% by mass, 3.0 to 15% by mass, 3.1 to 14% by mass, 3.2 to 13% by mass, 3.3 to 12% by mass, 3.4 to 11% by mass, 3.5 to 11% by mass, or the like.

The spray liquid preferably further contains erythritol in addition to HPC or HPMC. As shown in Example 2 described later, by adding erythritol to the spray liquid containing HPC or HPMC, erythritol granules capable of producing tablets having higher hardness can be produced.

The concentration of the erythritol in the spray liquid can be a maximum amount of erythritol capable of being dissolved in the liquid, and specifically, it can be less than 35% by mass. The weight ratio between the erythritol (ERT) and HPC is preferably either the following (a) or (b). The weight ratio between ERT and HPMC is preferably either the following (c) or (d):

(a) more than 2.5 parts by weight and less than 30 parts by weight of HPC based on 33 parts by weight of ERT,

(b) more than 3.3 parts by weight and less than 35 parts by weight of ERT based on 5 parts by weight of HPC,

(c) more than 2.5 parts by weight and less than 20 parts by weight of HPMC based on 33 parts by weight of ERT,

(d) more than 0 part by weight and less than 35 parts by weight of ERT based on 5 parts by weight of HPMC.

As shown in Example 3 and Example 4 described later, by determining the content ratio between HPC or HPMC and ERT in the spray liquid to any one of the above (a) to (d), erythritol granules capable of producing tablets having higher hardness can be produced.

As the granulation apparatus in the granulation step, for example, a batch fluidized bed granulator, such as normal fluidized bed granulator, forced circulation type fluidized bed granulator or spouted bed type granulator, or a continuous fluidized bed granulator, such as box type continuous fluidized bed granulator or cylindrical continuous fluidized bed granulator, can be used. Regarding the position of a spray nozzle for the spray liquid in the granulation apparatus, for example, any of a bottom spray system, a top spray system and a tangent spray system may be used. The granulation conditions can be appropriately determined according to the charge of erythritol, the desired properties of the erythritol granules, etc., and for example, the hot air inlet temperature can be set at 60 to 100° C., the air flow rate can be set to 0.4 to 0.8 m³/min, and the spray pressure of the spray liquid can be set at 0.1 to 0.3 MPa.

The method for producing tablets according to the present invention comprises a tableting step of tableting a mixture of the erythritol granules according to the present invention and a medicinal ingredient or a food ingredient by a direct tableting method. In this tableting step, if a mixture of the erythritol granules and the medicinal ingredient is tableted, a medicine or a quasi-drug in the dosage form of tablets can be produced, and if a mixture of the erythritol granules and the food ingredient is tableted, food and drink in the form of tablets, such as confectionary (tablet confectionary) or supplement, can be produced.

The mixture in the tableting step may contain substances other than the erythritol granules and the medicinal ingredient or the food ingredient as long as the features of the present invention are not impaired. Examples of such substances include lubricants and binders to improve processing characteristics, and food additives and pharmaceutical additives to improve flavor of tablets, palatability and preservability. Examples of the lubricants include magnesium stearate, glycerol fatty acid ester, sorbitan fatty acid ester and sucrose fatty acid ester.

The present invention will be described based on the examples hereinafter. The technical scope of the present invention is not limited to the features shown by these examples.

### EXAMPLES

### <Test Method>

Unless otherwise noted, Examples were carried out by the following methods (1) to (4). In Examples, “%” means “% by mass” unless otherwise noted. Erythritol is sometimes written as “ERT”.

(1) Erythritol and binder

As powdery erythritol, “Erythritol 100M (white powder, Japanese Pharmaceutical Excipients) (B FOOD SCIENCE CO., LTD)” was used. As binders, those shown in Table 1 were used.

TABLE 1

| | | Hydroxypropyl | Carboxymethyl | | |
|---|---|---|---|---|---|
| | Hydroxypropyl | methylcellulose | cellulose | Polyvinylpyrrolidone | Polyvinyl |
| Binder | cellulose (HPC) | (HPMC) | sodium (CMC) | (PVP) | alcohol (PVA) |
| Product | HPC SSL SFP | TC-5 | SUNROSE | Kollidon90F | Kuraray Poval |
| name | | | | | PVA403 |
| Manufacturer | Nippon Soda | Shin-Etsu | Nippon Parer | BASF | Kuraray Co., |
| | Co., Ltd. | Chemical Co., | Industries Co., | | Ltd. |
| | | Ltd. | Ltd. | | |

### (2) Granulation Method

Production of erythritol in the form of granules (erythritol granules, ERT granules) from powdery erythritol was carried out by a fluidized bed granulation method. That is to say, in a granulation apparatus “Multiplex FD-MP-01ND (Powrex Corporation)”, powdery erythritol was placed, and granulation was carried out while spraying a spray liquid at a hot air inlet temperature of 80° C., an air flow rate of 0.6 m³/min and a spray pressure of 0.2 MPa. As the spray liquid, a solution obtained by dissolving a binder and/or erythritol in water was used.

### (3) Tableting Method

Using the ERT granules, tablets were produced by a direct tableting method. That is to say, to 100 parts by weight of the ERT granules, 1 part by weight of magnesium stearate was added as a lubricant, and then, they were placed in a desktop type one-shot tableting machine “MINIPRESS MII (RIVA S.A.)” and compression molded into tablet shape at a tableting pressure of 5.0 to 6.0 kN. The tablet had a size of a diameter of 8 mm, and the weight of one tablet was 20 mg. That is to say, by applying a tableting pressure of 5.0 to 6.0 kN to a tablet area (π×0.4 cm×0.4 cm≈0.5 cm²), tablets were produced.

### (4) Evaluation Item and Evaluation Method

Regarding the ERT granules and the tablets produced, the following items [4-1] to [4-5] were evaluated. A list of the evaluation items and evaluation criteria is set forth in Table 2.

TABLE 2

| | Evaluation item | | | | |
|---|---|---|---|---|---|
| | Adhesion | | | | |
| | of spray | | Granule | | |
| | liquid | | diameter | | |
| Abbreviation | to the | Fluidity | of ERT | | Tablet |
| for | opening | of ERT | granules | Tablet | hardness |
| evaluation | of nozzle | granules | (μm) | formability | (kgf) |
| ⊚ | | (Not less | | | |
| | | than 70 and | | | |
| | | not more | | | |
| | | than 100) | | | |
| | | good | | | |
| ◯ | Not | (Not less | (Not less | Yes | (Not less |
| | observed | than 60 | than 50 | | than 4.5) |
| | | and less | and less | | prefer- |
| | | than 70) | than 250) | | able |
| | | moderate | preferable | | |
| Δ | Somewhat | (Not less | | | (Not less |
| | observed | than 40 and | | | than 3.5 |
| | | less than 60) | | | and less |
| | | somewhat | | | than 4.5) |
| | | poor | | | suitable |
| X | Observed | (Not less | (Less than | No | (Less |
| | | than 0 and | 50 or Not | | than 3.5) |
| | | less than | less than | | unsuit- |
| | | 40) | 250) | | able |
| | | poor | unsuitable | | |

### [4-1] Adhesion of Spray Liquid to the Opening of Nozzle

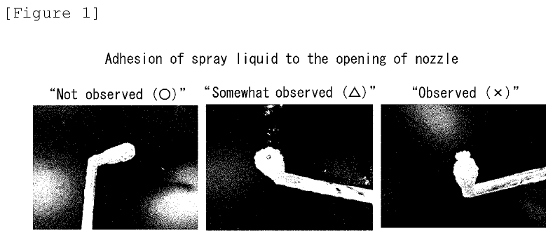

In the production of the ERT granules, whether the spray liquid adhered to the opening of nozzle for spraying the spray liquid in the granulation apparatus or not was visually confirmed. As shown in FIG. 1, a case where any adhesion mark of the spray liquid was not visually observed at the opening of nozzle was evaluated as “not observed (abbreviation: ◯)”; a case where an adhesion mark of a small amount of the spray liquid was visually observed was evaluated as “somewhat observed (abbreviation: A)”; and a case where an adhesion mark of a large amount of the spray liquid was visually observed was evaluated as “observed (abbreviation: x)”

### [4-2] Fluidity of ERT Granules

The degree of fluidity of the ERT granules was evaluated by calculating a fluidity index based on the powder property evaluation criteria of Carr. That is to say, using a powder characteristics evaluation apparatus “Powder Tester PT-X (Hosokawa Micron Corporation)”, “apparent specific gravity”, “degree of compression”, “angle of repose”, “spatula angle” and “degree of aggregation” were measured. Based on the measured values, respective indexes were calculated, and then the indexes were summed up to determine a fluidity index. When the fluidity index was not less than 70 and not more than 100, the degree of fluidity was evaluated as “good (abbreviation: ⊙)”; when the fluidity index was not less than 60 and less than 70, the degree of fluidity was evaluated as “moderate (abbreviation: ◯)”; when the fluidity index was not less than 40 and less than 60, the degree of fluidity was evaluated as “somewhat poor (abbreviation: Δ)”; and when the fluidity index was not less than 0 and less than 40, the degree of fluidity was evaluated as “poor (abbreviation: x)”.

### [4-3] Granule Diameter of ERT Granules

Using an Electromagnetic Micro Vibro Sifter M-2 (Tsutsui Scientific Instruments Co., Ltd.), a granule diameter distribution of the ERT granules was measured. Based on the measured value, a median diameter (d50) was calculated. When d50 was not less than 50 μm and less than 250 μm, the granules were evaluated as “preferable as granules for tableting (abbreviation: ◯)”; and when d50 was less than 50 μm or not less than 250 μm, the granules were evaluated as “unsuitable as granules for tableting (abbreviation: x)”

### [4-4] Tablet Formability

Regarding the tablets produced by using the ERT granules, occurrence of a “phenomenon where upper and lower parts of a tablet peel (capping)”, a “phenomenon where a part of a tablet adheres to a mallet and the part of the tablet peels (sticking)”, and a “phenomenon where a middle part of a tablet peels in layers (lamination)” was visually confirmed. A case where none of the phenomena were visually observed was evaluated as “there is tablet formability (0)”; and a case where one or more of the phenomena were visually observed was evaluated as “there is no tablet formability (x)”.

### [4-5] Tablet Hardness

A tablet hardness of the tablets produced by using the ERT granules was measured by a Monsanto Hardness Tester (Minato Medical Corporation). A tablet hardness of not less than 4.5 kilogram-force (kgf) was evaluated as “preferable (0)” as a hardness of a product; a tablet hardness of not less than 3.5 kgf and less than 4.5 kgf was evaluated as “suitable (A)”; and a tablet hardness of less than 3.5 kgf was evaluated as “unsuitable (x)”.

### <Example 1> Study of Binder

Hydroxypropyl cellulose (HPC), hydroxypropyl methylcellulose (HPMC), polyvinylpyrrolidone (PVP) and polyvinyl alcohol (PVA) were each dissolved in water in such a manner that each final concentration became 5%. A CMC solution having a concentration of 5% had such a high viscosity that it was not able to be sprayed, and therefore, CMC was dissolved in water in such a manner that the concentration became 1%. Using these solutions as spray liquids, ERT granules were produced to obtain ERT granules Nos. 1 to 5, and then tablets were produced and evaluated. The results are set forth in Table 3.

TABLE 3

| Sample No. | | No. 1 | No. 2 | No. 3 | No. 4 | No. 5 |
|---|---|---|---|---|---|---|
| Compounding | Type of binder | HPC | HPMC | CMC | PVP | PVA |
| | Binder concentration | 5 | 5 | 1 | 5 | 5 |
| | in spray liquid (%) | | | | | |
| | Binder concentration | 3.61 | 3.61 | 0.74 | 3.61 | 3.61 |
| | in ERT granules (%) | | | | | |
| Evaluation | Adhesion of | ○ | ○ | Δ | Δ | Δ |
| result | spray liquid | | | | | |
| | Fluidity of | ○ | ○ | ○ | ○ | ○ |
| | ERT granules | | | | | |
| | Granule diameter of | ○(113.7) | ○(111.6) | ×(429.3) | ○(162.4) | ○(137.2) |
| | ERT granules (μm) | | | | | |
| | Tablet formability | ○ | ○ | × | ○ | ○ |
| | Tablet hardness (kgf) | Δ(3.8 ± 0.4) | ○(4.7 ± 0.3) | ×(1.9 ± 0.1) | ×(3.4 ± 0.5) | ×(2.6 ± 0.1) |

As shown in Table 3, in each case of Nos. 3, 4 and 5 (using CMC, PVP and PVA, respectively), adhesion of the spray liquid to the opening of nozzle was somewhat observed, and the tablet hardness was low and unsuitable as a hardness of a product. In the case of No. 3 (using CMC), the granule diameter of the ERT granules is too large and unsuitable for granules for tableting, and there was no tablet formability. On the other hand, in each case of Nos. 1 and 2 (using HPC and HPMC, respectively), adhesion of the spray liquid to the opening of nozzle was not observed, the granule diameter of the ERT granules was preferable for granules for tableting, there was tablet formability, and the tablet hardness was a value preferable or suitable as a hardness of a product. From this result, it has become apparent that by adding HPC or HPMC, erythritol granules capable of producing tablets by a direct tableting method can be obtained.

### <Example 2> Effect Due to Compounding of Erythritol in Spray Liquid

Powdery erythritol was dissolved in water in such a manner that the final concentration became 33%, thereby obtaining a spray liquid No. 6. Moreover, HPC, HPMC, PVP and PVA were each dissolved in water in such a manner that each final concentration became 5%, and CMC was dissolved in water in such a manner that the final concentration became 1%, and then powdery erythritol was added to each of the resulting liquids in such a manner that the final concentration became 33%, thereby obtaining spray liquids Nos. 7 to 11. Using these spray liquids, ERT granules were produced to obtain ERT granules Nos. 6 to 11, and then tablets were produced and evaluated. The results are set forth in Table 4. In Table 4, the results of Nos. 1 to 5 of Example 1 are also set forth together for comparison.

TABLE 4

| Sample No. | | | No. 6 | No. 7 | No. 1 | No. 8 | No. 2 |
|---|---|---|---|---|---|---|---|
| Compounding | Type of binder | | None | HPC | HPC | HPMC | HPMC |
| | Binder concentration in spray liquid (%) | | 0 | 5 | 5 | 5 | 5 |
| | Binder concentration in ERT granules (%) | | 0 | 2.91 | 3.61 | 2.91 | 3.61 |
| | ERT concentration in spray liquid (%) | | 33 | 33 | 0 | 33 | 0 |
| Evaluation | Adhesion of spray liquid | | Δ | ○ | ○ | ○ | ○ |
| result | Fluidity of ERT granules | | Δ | ○ | ○ | ○ | ○ |
| | Granule diameter of ERT granules (μm) | | ○(124.5) | ○(108.7) | ○(113.7) | ○(94) | ○(111.6) |
| | Tablet formability | | × | ○ | ○ | ○ | ○ |
| | Tablet hardness (kgf) | | — | ○(4.7 ± 0.6) | Δ(3.8 ± 0.4) | ○(5.4 ± 0.5) | ○(4.7 ± 0.3) |
| Sample No. | | No. 9 | No. 3 | No. 10 | No .4 | No. 11 | No. 5 |
| Compounding | Type of binder | CMC | CMC | PVP | PVP | PVA | PVA |
| | Binder concentration in spray | 1 | 1 | 5 | 5 | 5 | 5 |
| | liquid (%) | | | | | | |
| | Binder concentration in ERT | 0.60 | 0.74 | 2.91 | 3.61 | 2.91 | 3.61 |
| | granules (%) | | | | | | |
| | ERT concentration in spray | 32 | 0 | 33 | 0 | 33 | 0 |
| | liquid (%) | | | | | | |
| Evaluation | Adhesion of spray liquid | Δ | Δ | × | Δ | Δ | Δ |
| result | Fluidity of ERT granules | Δ | ○ | Δ | ○ | Δ | ○ |
| | Granule diameter of ERT | ×(357.7) | ×(429.3) | ×(325.4) | ○(162.4) | ○(122.4) | ○(137.2) |
| | granules (μm) | | | | | | |
| | Tablet formability | × | × | ○ | ○ | ○ | ○ |
| | Tablet hardness (kgf) | ×(1.7 ± 0.2) | ×(1.9 ± 0.1) | ○(9.5 ± 00.4) | × (3.4 ± 0.5) | × (3.3 ± 0.2) | × (2.6 ± 0.1) |

As shown in Table 4, in each case of No. 7 and No. 8, adhesion of the spray liquid to the nozzle opening was not observed, the fluidity of the ERT granules was moderate, the granule diameter of the ERT granules was preferable for granules for tableting, there was tablet formability, and the tablet hardness was preferable as a hardness of a product. In comparison between No. 7 and No. 1, No. 7 had a higher tablet hardness. Likewise, in comparison between No. 8 and No. 2, No. 8 had a higher tablet hardness. That is to say, it has become apparent that by adding erythritol to a spray liquid containing HPC or HPMC, erythritol granules capable of producing tablets having higher hardness can be produced. From this result, it has become apparent that by carrying out granulation while spraying a spray liquid containing erythritol in addition to HPC or HPMC, erythritol granules preferable for producing tablets by a direct tableting method can be obtained.

On the other hand, in the case of No. 6, there was no tablet formability, and tableting was impossible. That is to say, it has become apparent that when granulation is carried out while spraying a spray liquid containing erythritol only but not containing HPC or HPMC, erythritol granules suitable for producing tablets by a direct tableting method cannot be obtained.

In the case of No. 9, the granule diameter of the ERT granules was unsuitable, the granules had no tablet formability, and the tablet hardness was unsuitable. In the case of No. 10, adhesion of the spray liquid to the opening of nozzle was observed, and the granule diameter of the ERT granules was unsuitable. In the case of No. 11, the tablet hardness was unsuitable. That is to say, it has become apparent that even if erythritol is added to a spray liquid containing a binder of CMC, PVP or PVA, erythritol granules suitable for producing tablets by a direct tableting method cannot be obtained.

### <Example 3> Electron Microscopic Observation

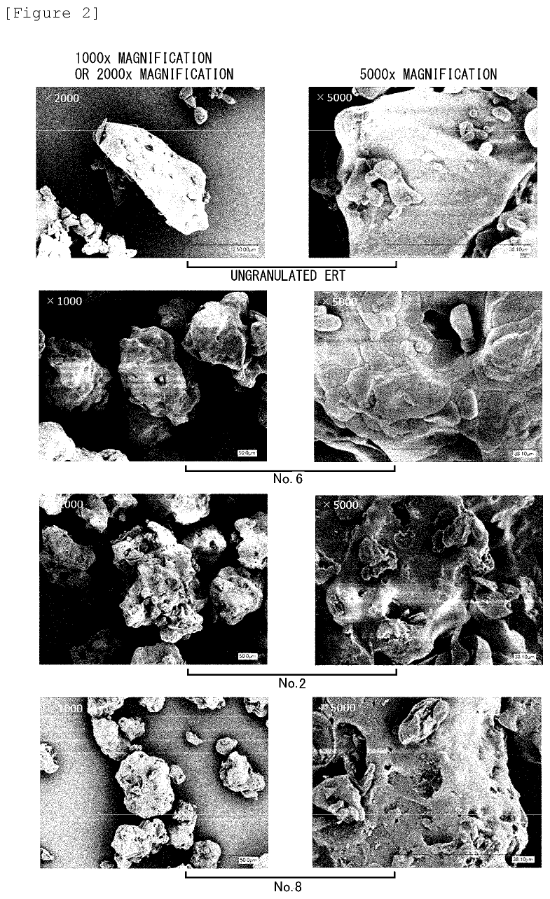

Ungranulated powdery erythritol (ungranulated ERT), and ERT granules of No. 2 of Example 1 and No. 6 and No. 8 of Example 2 were observed by a scanning type electron microscope at a magnification of 1000× or 2000× and 5000×. The observation images are shown in FIG. 2. For reference, compounding in each sample and evaluation results of the ERT granules are set forth again in Table 5.

TABLE 5

| | | Sample No. | | | |
|---|---|---|---|---|---|
| | | Ungranulated | | | |
| | | ERT | No. 2 | No. 6 | No. 8 |
| Compounding | Type of binder | | HPMC | None | HPMC |
| | Binder concentration in | | 5 | 0 | 5 |
| | spray liquid (%) | | | | |
| | Binder concentration in | | 3.61 | 0 | 2.91 |
| | ERT granules (%) | | | | |
| | ERT concentration in | | 0 | 33 | 33 |
| | spray liquid (%) | | | | |
| Evaluation | Adhesion of spray liquid | | ○ | Δ | ○ |
| result | Fluidity of ERT granules | | ○ | Δ | ○ |
| | Granule diameter of | | ○(111.6) | ○(124.5) | ○(94) |
| | ERT granules (μm) | | | | |
| | Tablet formability | | ○ | x | ○ |
| | Tablet hardness (kgf) | | ○(4.7 ± 0.3) | — | ○(5.4 ± 0.5) |

As shown in FIG. 2, the ERT granules of No. 2 and No. 8 each had a structure of many pores (porous structure) as compared with the ungranulated ERT and the ERT granules of No. 6. From this result, it has become apparent that the erythritol granules of the present invention each have a porous structure as compared with the erythritol granules containing no binder or the ungranulated powdery erythritol. It has been thought that owing to this porous structure, high tabletability, such as high formability and binding property in the production of tablets, and high hardness of the tablets produced have been attained in the erythritol granules of the present invention.

### <Example 4> Average Yield Pressure of ERT Granules

In general, during tableting, as a pressure (compression pressure) is applied to a powder layer to compress the layer, the volume of the powder layer decreases. In the compression initial stage, voids in the powder decrease, and thereafter, plastic deformation of powder granules takes place, and this process can be divided into the following three stages.

First stage: compression initial stage; rearrangement and breakage of secondary granules take place, and the secondary granules become primary granules.

Second stage: plastic deformation of the primary granules takes place.

Third stage: breakage and rearrangement of the primary granules also take place in addition to the plastic deformation of the primary granules.

In the stages in which the powder layer displays plastic deformation (second stage, third stage), a plot (Heckel plot) of a compression pressure (P) against a natural logarithm of a reciprocal number of a void ratio (c) becomes a straight line, and the equation of the straight line is called Heckel equation, and expressed by In(1/ε)=KP+A (K and A are constants) (Heckel, R. W., Density-pressure Relationships in Powder Compaction, Trans. Met. Soc. AIME, 221, 671(1961); Heckel, E. W., An analysis of Powder Compaction Phenomena, Trans. Met. Soc. AIME, 221, 1001 (1961)). Here, K is a slope of a linear portion of the Heckel plot, and a reciprocal number of K denotes an average yield pressure (Py). The average yield pressure is an indication indicating ease of plastic deformation of the powder layer, and it can be judged that as its value is lowered, the powder layer easily undergoes plastic deformation, that is, the tablet production is easy.

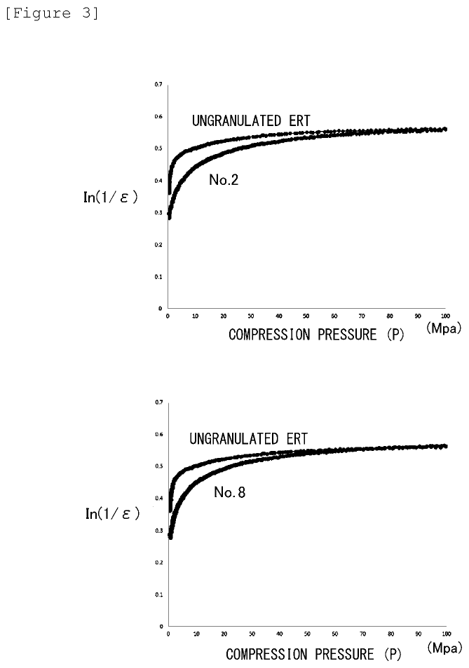

Average yield pressures of the ungranulated powdery erythritol (ungranulated ERT), and the ERT granules of No. 2 of Example 1 and No. 8 of Example 2 were determined. Specifically, a sample obtained by adding 1.6 mg of magnesium stearate to 160 mg of the ungranulated ERT or the ERT granules of No. 2 or No. 8 was filled in mortar (diameter: 8 mm) of a precision universal tester (AUTOGRAPH, Shimadzu Corporation), and the sample was compressed at a compression rate of 10 mm/min and a pressure of 0 to 100 MPa. From the position of the mallet at each compression pressure, the volume of the powder layer was calculated, and based on the resulting value, a void ratio (c) (%) of the powdery layer was calculated. Next, the compression pressure (P) is plotted as abscissa and the natural logarithm (In(1/ε)) of a reciprocal number of the void ratio of the powder layer as ordinate to make a Heckel plot. Based on the shape of the Heckel plot (FIG. 3) thus made, the ranges of the compression pressures corresponding to the first to third stages were specified, and using the equation 1 {In(1/ε)=KP+A (K and A are constants)} and the equation 2 {Py=1/K}, an average yield pressure (Py) in each stage was calculated. The results are set forth in Table 6.

TABLE 6

| | First stage | | Second stage | | Third stage | |
|---|---|---|---|---|---|---|
| | Compression | Average yield | Compression | Average yield | Compression | Average yield |
| | pressure: P | pressure: Py | pressure: P | pressure: Py | pressure: P | pressure: Py |
| Sample | (MPa) | (MPa) | (MPa) | (MPa) | (MPa) | (MPa) |
| Ungranulated | <2 | 19 | 5-30 | 486 | 30-100 | 2941 |
| ERT | | | | | | |
| No. 2 | <2 | 28 | 12-30 | 332 | 30-100 | 1441 |
| No. 8 | <2 | 27 | 12-30 | 324 | 30-100 | 1441 |

As shown in Table 6, the average yield pressures of No. 2 and No. 3 in the second stage that was a stage in which plastic deformation of the powder granules took place were 332 MPa and 324 MPa, respectively, and were markedly low as compared with 486 MPa of the ungranulated ERT. Likewise, the average yield pressures of No. 2 and No. 3 in the third stage that was a stage in which breakage and rearrangement of the powder granules took place in addition to the plastic deformation were each 1441 MPa and were markedly low as compared with 2941 MPa of the ungranulated ERT. From this result, it has become apparent that as compared with the ungranulated powdery erythritol, the erythritol granules of the present invention having a porous structure have a low average yield pressure in a stage in which the powder layer displays plastic deformation, that is, they are granules which easily undergo plastic deformation and have ease of tablet production. From this result, it has become apparent that the average yield pressure of the erythritol granules of the present invention in the range of 30 to 100 MPa under the compression conditions of Example 4 is around 1400 MPa, or not less than 1200 MPa and not more than 1600 MPa, not less than 1000 MPa and not more than 1800 MPa, not less than 800 MPa and not more than 2000 MPa, not less than 600 MPa and not more than 2200 MPa, not less than 400 MPa and not more than 2400 MPa, not less than 200 MPa and not more than 2600 MPa, not more than 2800 MPa, or not more than 2941 MPa.

### <Example 5> Amount of Binder Compounded

### (1) Amount of HPC Compounded

HPC was dissolved in water in such a manner that the final concentration became 1 to 30%, and powdery erythritol was added in such a manner that the final concentration became 33%. Using these liquids as spray liquids, ERT granules were produced, and thereafter, tablets were produced and evaluated. The results are set forth in Table 7.

TABLE 7

| Compounding | Type of binder | HPC | HPC | HPC | HPC | HPC | HPC |
|---|---|---|---|---|---|---|---|
| | Binder concentration | 1 | 2.5 | 5 | 10 | 20 | 30 |
| | in spray liquid (%) | | | | | | |
| | Binder concentration | 0.60 | 1.48 | 2.91 | 5.66 | 10.71 | 15.25 |
| | in ERT granules (%) | | | | | | |
| | ERT concentration | 33 | 33 | 33 | 33 | 33 | 33 |
| | in spray liquid (%) | | | | | | |
| Evaluation | Adhesion of spray | ○ | Δ | ○ | Δ | Δ | Glanulation |
| result | liquid | | | | | | was |
| | Fluidity of ERT | ○ | ○ | ○ | Δ | ○ | impossible |
| | granules | | | | | | |
| | Granule diameter of | ○(75.5) | ○(69.1) | ○(108.7) | ○(138.3) | ○(109) | |
| | ERT granules (μm) | | | | | | |
| | Tablet formability | ○ | ○ | ○ | ○ | ○ | |
| | Tablet hardness (kgf) | ×(0.8 ± 0.1) | ×(2.8 ± 0.2) | ○(4.7 ± 0.6) | ○(5.1 ± 0.5) | ○(8.2 ± 0.1) | |

As shown in Table 7, when the HPC concentrations in the spray liquids were 5%, 10% and 20% (the HPC concentrations in the ERT granules were 2.91%, 5.66% and 10.71%), adhesion of the spray liquid to the nozzle opening was not observed or somewhat observed, the fluidity of the ERT granules was moderate or somewhat poor, the granule diameter of the ERT granules was preferable for granules for tableting, there was tablet formability, and the tablet hardness was preferable as a harness of a product. On the other hand, when the HPC concentrations in the spray liquids were 1% and 2.5% (the HPC concentrations in the ERT granules were 0.60% and 1.48%), the tablet hardness was low and unsuitable as a hardness of a product. When the HPC concentration in the spray liquid was 30% (the HPC concentration in the ERT granules was 15.25%), the viscosity of the spray liquid was so high that the spray liquid was not able to be sprayed, and the granulation was impossible. From this result, it has become apparent that when the HPC concentration in the ERT granules containing HPC is set to more than 1.48% and less than 15.25%, or when the HPC concentration in the spray liquid is set to less than 30% and the amount of HPC is set to more than 2.5 parts by weight and less than 30 parts by weight based on 33 parts by weight of the erythritol, erythritol granules preferable for producing tablets by a direct tableting method can be obtained.

### (2) Amount of HPMC Compounded

HPMC was dissolved in water in such a manner that the final concentration became 1 to 20%, and powdery erythritol was added in such a manner that the final concentration became 33%. Using these liquids as spray liquids, ERT granules were produced, and thereafter, tablets were produced and evaluated. The results are set forth in Table 8.

TABLE 8

| Compounding | Type of binder | HPMC | HPMC | HPMC | HPMC | HPMC |
|---|---|---|---|---|---|---|
| | Binder concentration in | 1 | 25 | 5 | 10 | 20 |
| | spray liquid (%) | | | | | |
| | Binder concentration in | 0.60 | 1.48 | 2.91 | 5.66 | 10.71 |
| | ERT granules (%) | | | | | |
| | ERT concentration in | 33 | 33 | 33 | 33 | 33 |
| | spray liquid (%) | | | | | |
| Evaluation | Adhesion of spray liquid | ○ | ○ | ○ | Δ | Glanulation |
| result | Fluidity of ERT granules | ○ | ○ | ○ | ○ | was |
| | Granule diameter of | ○(88.5) | ○(98.8) | ○(94) | ○(152.7) | impossible |
| | ERT granules (μm) | | | | | |
| | Tablet formability | ○ | × | ○ | ○ | |
| | Tablet hardness (kgf) | ×(3.4 ± 0.7) | ×(1.2 ± 0.2) | ○(5.4 ± 0.5) | ○(5.3 ± 0.3) | |

As shown in Table 8, when the HPMC concentrations in the spray liquids were 5% and 10% (the HPMC concentrations in the ERT granules were 2.91% and 5.66%), adhesion of the spray liquid to the nozzle opening was not observed or somewhat observed, the fluidity of the ERT granules was moderate, the granule diameter of the ERT granules was preferable for granules for tableting, there was tablet formability, and the tablet hardness was preferable as a hardness of a product. On the other hand, when the HPMC concentrations in the spray liquids were 1% and 2.5% (the HPMC concentrations in the ERT granules were 0.60% and 1.48%), the tablet hardness was low and unsuitable as a hardness of a product. When the HPMC concentration in the spray liquid was 20% (the HPMC concentration in the ERT granules was 10.71%), the viscosity of the spray liquid was so high that the spray liquid was not able to be sprayed, and the granulation was impossible. From this result, it has become apparent that when the HPMC concentration in the ERT granules containing HPMC is set to more than 1.48% and less than 10.71%, or when the HPMC concentration in the spray liquid is set to less than 20% and the amount of HPMC is set to more than 2.5 parts by weight and less than 20 parts by weight based on 33 parts by weight of the erythritol, erythritol granules preferable for producing tablets by a direct tableting method can be obtained.

### <Example 6> Amount of Erythritol Compounded in Spray Liquid

### (1) Spray Liquid Containing HPC

HPC was dissolved in water in such a manner that the final concentration became 5%, and powdery erythritol was added in such a manner that the final concentration became 3.3 to 35%. Using these liquids as spray liquids, ERT granules were produced, and thereafter, tablets were produced and evaluated. The results are set forth in Table 9.

TABLE 9

| Compounding | Type of binder | HPC | HPC | HPC | HPC | HPC |
|---|---|---|---|---|---|---|
| | Binder concentration in | 5 | 5 | 5 | 5 | 5 |
| | spray liquid (%) | | | | | |
| | ERT concentration in | 3.3 | 6.7 | 16.7 | 33.3 | 35 |
| | spray liquid (%) | | | | | |
| Evaluation | Adhesion of spray liquid | Δ | ○ | ○ | ○ | Glanulation |
| result | Fluidity of ERT granules | ○ | Δ | Δ | ○ | was |
| | Granule diameter of ERT | ○(131.9) | ○(101.2) | ○(114.9) | ○(108.7) | impossible |
| | granules (μm) | | | | | |
| | Tablet formability | ○ | ○ | ○ | ○ | |
| | Tablet hardness (kgf) | ×(2.6 ± 0.3) | ○(5.6 ± 0.5) | ○(4.6 ± 0.5) | ○(4.7 ± 0.6) | |

As shown in Table 9, when the ERT concentrations in the spray liquids were 6.7%, 16.7% and 33.3%, adhesion of the spray liquid to the nozzle opening was not observed, the fluidity of the ERT granules was moderate or somewhat poor, the granule diameter of the ERT granules was preferable for granules for tableting, there was tablet formability, and the tablet hardness was preferable as a hardness of a product. On the other hand, when the ERT concentration in the spray liquid was 3.3%, there was no tablet formability. When the ERT concentration in the spray liquid was 35%, the erythritol was not completely dissolved, so that the concentration did not become homogeneous, and the granulation was impossible. From this result, it has become apparent that regarding the ERT granules containing HPC, when the erythritol concentration in the spray liquid is set to less than 35% and the amount of erythritol is set to more than 3.3 parts by weight and less than 35 parts by weight based on 5 parts by weight of HPC, erythritol granules preferable for producing tablets by a direct tableting method can be obtained.

### (2) Spray Liquid Containing HPMC

HPMC was dissolved in water in such a manner that the final concentration became 5%, and powdery erythritol was added in such a manner that the final concentration became 3.3 to 35%. Using these liquids as spray liquids, ERT granules were produced, and thereafter, tablets were produced and evaluated. The results are set forth in Table 10.

TABLE 10

| Compounding | Type of binder | HPMC | HPMC | HPMC | HPMC | HPMC |
|---|---|---|---|---|---|---|
| | Binder concentration in | 5 | 5 | 5 | 5 | 5 |
| | spray liquid (%) | | | | | |
| | ERT concentration in | 3.3 | 6.7 | 16.7 | 33.3 | 35 |
| | spray liquid (%) | | | | | |
| Evaluation | Adhesion of spray liquid | ○ | ○ | ○ | ○ | Glanulation |
| result | Fluidity of ERT granules | ○ | ○ | Δ | ○ | was |
| | Granule diameter of | ○(107.3) | ○(131.1) | ○(104.8) | ○(94) | imrossible |
| | ERT granules (μm) | | | | | |
| | Tablet formability | ○ | ○ | ○ | ○ | |
| | Tablet hardness (kgf) | ○(5.8 ± 0.5) | ○(5.6 ± 0.2) | ○(4.8 ± 0.4) | ○(5.4 ± 0.5) | |

As shown in Table 10, when the ERT concentrations in the spray liquids were 3.3%, 6.7%, 16.7% and 33.3%, adhesion of the spray liquid to the opening of nozzle was not observed, the fluidity of the ERT granules was moderate or somewhat poor, the granule diameter of the ERT granules was preferable for granules for tableting, there was tablet formability, and the tablet hardness was preferable as a hardness of a product. On the other hand, when the ERT concentration in the spray liquid was 35%, the erythritol was not completely dissolved, so that the concentration did not become homogeneous, and the granulation was impossible. From this result, it has become apparent that regarding the ERT granules containing HPMC, when the erythritol concentration in the spray liquid is set to less than 35% and the amount of erythritol is set to more than 0 part by weight and less than 35 parts by weight based on 5 parts by weight of HPMC, erythritol granules preferable for producing tablets by a direct tableting method can be obtained.

### <Example 7> Production of Supplement

HPMC was dissolved in water in such a manner that the final concentration became 5%, and powdery erythritol was added in such a manner that the final concentration became 33.3%. Using this liquid as a spray liquid, ERT granules were produced. Subsequently, the ERT granules, N-acetylglucosamine and magnesium stearate (lubricant) were mixed in ratios of 49%, 50% and 1%, respectively, then the mixture was tableted by the method described in the test method (3) to produce a supplement, and the tablet formability and the tablet hardness were evaluated. As Comparative Example, a supplement was produced by using powdery erythritol instead of the ERT granules and performing tableting in the same manner as above, and the supplement was evaluated. The results are set forth in Table 11.

TABLE 11

| | | Ex. 7 | Comp. Ex. |
|---|---|---|---|
| Compounding | Erythritol | 0 | 49 |
| (%) | ERT granules | 49 | 0 |
| | N-acetylglucosamine | 50 | 50 |
| | Magnesium stearate | 1 | 1 |
| Evaluation | Tablet formability | ○ | x |
| result | Tablet hardness (kgf) | ○(4.7 ± 0.7) | x |

As shown in Table 11, in the case of the supplement of Example 7, there was tablet formability, and the tablet hardness was preferable as a hardness of a product. When the supplement was eaten, there was refreshing and good-quality sweetness of erythritol, and bitterness and astringency derived from N-acetylglucosamine were suppressed, so that this supplement tasted good. On the other hand, in the case of the supplement of the Comparative Example, there was no tablet formability, and the tablet hardness was unsuitable as a hardness of a product. From this result, it has become apparent that by using the erythritol granules of the present invention, tablets having sufficient hardness can be produced by a direct tableting method.

1. Erythritol granules comprising hydroxypropyl cellulose or hydroxypropyl methylcellulose, the granules having a property that when a sample obtained by adding 1.6 mg of magnesium stearate to 160 mg of the erythritol granules is filled in a mortar having a diameter of 8 mm and compressed at a compression rate of 10 mm/min and a pressure of 0 to 100 MPa, an average yield pressure in the range of 30 to 100 MPa is less than 2941 MPa.

2. Erythritol granules comprising more than 1.48% by mass and less than 15.25% by mass of hydroxypropyl cellulose or more than 1.48% by mass and less than 10.71% by mass of hydroxypropyl methylcellulose.

3. The erythritol granules according to claim 1, for producing tablets by a dry direct tableting method.

4. The erythritol granules according to claim 1, having a property that when tablets each having a diameter of 8 mm and weighing 200 mg per tablet are formed by adding 1 part by weight of magnesium stearate to 100 parts by weight of the erythritol granules and then tableting the mixture by a dry direct tableting method at a tableting pressure of 5.0 to 6.0 kN, a hardness of each of the tablets is not less than 3.5 kgf.

5. A method for producing erythritol granules, comprising a granulation step of spraying a spray liquid comprising hydroxypropyl cellulose and/or hydroxypropyl methylcellulose to erythritol powder while fluidizing or stirring the erythritol powder, and then drying.

6. The method for producing erythritol granules according to claim 5, wherein the granulation step is carried out by a fluidized bed granulation method.

7. The method for producing erythritol granules according to claim 5, wherein in the spray liquid, a concentration of the hydroxypropyl cellulose is more than 2.5% by mass and less than 30% by mass, or a concentration of the hydroxypropyl methylcellulose is more than 2.5% by mass and less than 20% by mass.

8. The method for producing erythritol granules according to claim 5, wherein the spray liquid further comprises erythritol.

9. The method for producing erythritol granules according to claim 8, wherein in the spray liquid, a concentration of the erythritol is less than 35% by mass, and a weight ratio between the erythritol and the hydroxypropyl cellulose is either the following (a) or (b), or a weight ratio between the erythritol and the hydroxypropyl methylcellulose is either the following (c) or (d):
(a) more than 2.5 parts by weight and less than 30 parts by weight of the hydroxypropyl cellulose based on 33 parts by weight of the erythritol,
(b) more than 3.3 parts by weight and less than 35 parts by weight of the erythritol based on 5 parts by weight of the hydroxypropyl cellulose,
(c) more than 2.5 parts by weight and less than 20 parts by weight of the hydroxypropyl methylcellulose based on 33 parts by weight of the erythritol,
(d) more than 0 part by weight and less than 35 parts by weight of the erythritol based on 5 parts by weight of the hydroxypropyl methylcellulose.

10. A method for producing tablets, comprising a tableting step of tableting a mixture of the erythritol granules according to claim 1 and a medicinal ingredient or a food ingredient by a dry direct tableting method.

11. A tablet comprising the erythritol granules according to claim 1 and a medicinal ingredient or a food ingredient.

12. The erythritol granules according to claim 2, for producing tablets by a dry direct tableting method.

13. The erythritol granules according to claim 2, having a property that when tablets each having a diameter of 8 mm and weighing 200 mg per tablet are formed by adding 1 part by weight of magnesium stearate to 100 parts by weight of the erythritol granules and then tableting the mixture by a dry direct tableting method at a tableting pressure of 5.0 to 6.0 kN, a hardness of each of the tablets is not less than 3.5 kgf.

14. A method for producing tablets, comprising a tableting step of tableting a mixture of the erythritol granules according to claim 2 and a medicinal ingredient or a food ingredient by a dry direct tableting method.

15. A tablet comprising the erythritol granules according to claim 2 and a medicinal ingredient or a food ingredient.
